(19) United States
(12) Patent Application Publication
(10) Pub. No.: US 2010/0056626 A1
(43) Pub. Date: Mar. 4, 2010

(54) COSMETIC PRODUCT FOR THE TREATMENT OF CICATRICIAL TISSUES

(76) Inventor: Nicolò Scuderi, Roma (IT)

Correspondence Address: MODIANO & ASSOCIATI, Via Meravigli, 16, Milano,, 20123, IT

(21) Appl. No.: 12/312,432

(22) PCT Filed: Nov. 17, 2006
(86) PCT No.: PCT/IT2006/000802

§ 371 Date: May 11, 2009

(51) Int. Cl. A61K 31/22; C07C 229/00; A61P 17/00

(52) U.S. Cl. 514/526; 560/172

(57) ABSTRACT

A method for cosmetic treatment of cicatricial tissues comprising the step of applying to the cicatricial tissue a cosmetic product which contains one or more cyanoacrylates.

The present invention relates to a cosmetic product for the treatment of cicatricial tissues obtained by using at least one cyanoacrylate.

### BACKGROUND OF THE INVENTION

The term “scar” references the portion of tissue that constitutes the closure of wounds and in particular of losses of small and/or large portions of tissues and organs. Scar tissue in fact forms at each interruption of the continuity of the skin (epidermis and dermis) as a consequence of a pathological or traumatic event.

If poor tissue healing occurs (for example, the tissues have closed in a way which compromises some characteristics such as elasticity, softness and/or aesthetic appearance), true problems linked thereto can arise: keloids and hypertrophic scars.

A keloid is a thick reddish formation, which in some cases can even be painful, can form at a scar and often affects a surface which is larger than the surface actually affected by the base scar; it is often caused by the presence of bacteria during the “maturation” period (from healing to the final stage) of the scar.

A hypertrophic scar is merely a scar whose tissues undergo a sort of thickening and reddening (as the etymology of the word “hypertrophic” suggests, this is indeed a scar in which an increase in volume of the tissue has occurred).

The state in which the scars appear, regardless of the nature of the lack of tissue that produced them, depends mainly on the type of tissue of the carrier individual and on their behavior during healing. Secondly, the kind of events that determined the scars and the protection to which they were subjected during the healing step constitute an important component in the formation of scars affected by various problems.

Keloids and hypertrophic scars can be improved aesthetically by means of substantially cosmetic treatments (application of appropriate products) and by surgery.

Surgical procedures can be performed effectively only after long periods of time have elapsed from the generating event, in order to operate on a surface which is by then stable and no longer subjected to transformations due to the final phases of healing.

In the first period, approximately the period from the tenth day after the generating event (be it traumatic or surgical) up to the complete maturation of the scar tissues, it is possible to use only treatments of a cosmetic type, including mainly massages, prolonged compression and/or bandaging or spreading with silicone and derivatives thereof.

Recently, there has been a greater diffusion of cosmetic treatments based on the use of silicone; this in fact ensures that the scar tissues remain in optimum temperature and humidity conditions and at the same time constitutes a layer for protecting tissues with respect to subsequent traumas (during the healing period).

The extreme mobility of silicone bandages and, more evidently in the case of silicone gels or the like, the fact that it is totally impossible to keep the tissues motionless constitute a severe problem of this type of treatment. Complete softness, further, does not ensure sufficient protection against impacts. Likewise, immersion of the scar protected with silicone can be dangerous, since the liquid infiltrates and reaches the scar tissues, compromising the effects of the treatment.

Further, treatment with silicone does not allow simultaneous compression of the tissues and in particular of the flaps of the wound, unless additional bandages arranged above said treatment are used.

### SUMMARY OF THE INVENTION

The aim of the present invention is to obtain a cosmetic product for the treatment of cicatricial tissues which is capable of immobilizing cicatricial tissues and the tissues that are directly proximate to them.

Within this aim, an object of the present invention is to obtain a cosmetic product for the treatment of cicatricial tissues with an antibacterial function on cicatricial tissues and on the tissues that are directly proximate to them.

Another object of the present invention is to obtain a cosmetic product for the treatment of cicatricial tissues which is capable of waterproofing completely cicatricial tissues and the tissues that are directly proximate to them.

Another object of the present invention is to obtain a cosmetic product for the treatment of cicatricial tissues which is capable of maintaining optimum levels of humidity of the cicatricial tissues and of the tissues that are directly proximate to them.

Another object of the present invention is to obtain a cosmetic product for the treatment of cicatricial tissues which is capable of maintaining optimum temperature levels of the cicatricial tissues and of the tissues that are directly proximate to them.

Another object of the present invention is to obtain a cosmetic product for the treatment of cicatricial tissues which is capable of creating a microenvironment which is favorable for healing and in which there is minimal exchange with the outside at the cicatricial tissues and at the tissues that are directly proximate to them.

Another object of the present invention is to obtain a cosmetic product for the treatment of cicatricial tissues which is capable of protecting from impacts cicatricial tissues and tissues which are directly proximate thereto, constituting a sort of mechanically rigid protective dome.

A further object of the present invention is to obtain a cosmetic product for the treatment of cicatricial tissues which is capable of dehydrating tissues during the heating caused by polymerization, eliminating thermolabile toxins and bacteria.

A still further object of the present invention is to provide a cosmetic product for the treatment of cicatricial tissues which has a low cost, is relatively simple to provide in practice and safe in application.

This aim and these and other objects, which will become better apparent hereinafter, are achieved by the present use of at least one cyanoacrylate to prepare a cosmetic product for the treatment of cicatricial tissues.

### DESCRIPTION OF THE PREFERRED EMBODIMENTS

Cyanoacrylics or cyanoacrylates (the chemical name of so-called “instant” adhesives) are known as commercial adhesives since the 1950s, being used initially as special adhesives for very particular niche applications. Their exceptional adhesive characteristics and ease of use have made them very well-known and widespread for applications on a wide variety of materials. Cyanoacrylics polymerize in a few seconds or at the most a few minutes. These resins are alkyl esters based on cyanoacrylic acid. Over the years, numerous cyanoacrylics have been synthesized, such as propyl, butyl, isobutyl, ethyl and methyl cyanoacrylate. Cyanoacrylic products generally are formulated without thickening agents and are therefore liquid with a viscosity comprised between 1 and 3 MPas/s (close to the viscosity of water). When thickening agents are added, viscosity can reach 2000 MPas/s or higher values, as in the case of gel or thixotropic products. These adhesives are easy to use because they do not tend to migrate or drip on the point of application; however, liquid adhesives, without any type of filler, are dosed better and more quickly. Once polymerized, they allow to obtain films of hardened product which are very thin with respect to more viscous ones, to the full benefit of performance and of better industrialization.

The use of at least one cyanoacrylate to prepare a cosmetic product for the treatment of cicatricial tissues consists in practice in providing a product which is suitable to be distributed uniformly on scars which are healing.

In particular, the application of the cosmetic product according to the invention consists in arranging it on a tissue lesion which has already healed: approximately, the product according to the invention can be applied starting from the tenth day after the event that produced the lesion (although this time interval can change, for example, depending on the conditions of the tissue lesion, on the environment in which healing occurs, and on the characteristics of the patient).

In practice, the cosmetic product contains, in addition to excipients of various kinds (and proposed for different purposes), a few examples of which are given hereinafter, at least one form of cyanoacrylate.

Among currently known forms of cyanoacrylate, the group of main interest is constituted by hydrocarbon derivatives of cyanoacrylate.

In particular, the hydrocarbon derivatives of cyanoacrylate which are potentially interesting comprise radicals with 1 to 12 carbon atoms, preferably 1 to 8 carbon atoms.

Likewise, embodiments which comprise hydrocarbon derivatives of cyanoacrylate, such as alkoxy-alkyl cyanoacrylates, for example methoxy isopropyl cyanoacrylate, can also be adapted for the use according to the invention.

For correct understanding of some of the formulations of practical interest of the cosmetic product according to the invention, it is necessary to list the embodiment that comprises a cyanoacrylic monomer of the type of methoxy propyl cyanoacrylate.

Likewise, other embodiments of particular practical interest comprise butyl cyanoacrylate, methyl cyanoacrylate, propyl cyanoacrylate, isobutyl cyanoacrylate, ethyl cyanoacrylate.

The cosmetic product according to the invention can further comprise other substances, with the substantial function of excipients, such as for example dilution solvents and/or substances for adjusting the viscosity and/or substances for adjusting thixotropy.

The application of the product to the scar consists in arranging at least one layer of product (subsequent ones, if any, will be superimposed on the first one) on the cicatricial tissues and on the tissues that lie proximate thereto, approximately not before the tenth day after the event that caused the lesion of the tissues.

In practice, the product is applied during the step known as maturation of the scar. In any case, the product is used conveniently after removing the suture stitches.

The arrangement by successive layers makes the polymerization reaction (a reaction of the exothermic type) less unpleasant for the patient, who feels heat without reaching discomfort caused by an actual burn: by arranging the layers one at a time, the reaction produces gradually limited amounts of heat due to the small amount of product that is distributed.

By way of example, successive applications should be performed at time intervals of approximately 5-7 days (in some cases, regions exposed to impacts and abrasions, it is necessary to reduce the extent of said intervals).

The distribution of superimposed layers further constitutes a sort of protective dome for cicatricial tissues, which especially in the first period after the event that caused the lesion of the tissues (as an indication, starting from the tenth day up to a few months later), by being particularly delicate, can suffer lesions even as a consequence of small impacts.

Advantageously, the cosmetic product for treating cicatricial tissues is capable of immobilizing cicatricial tissues and the tissues that are directly proximate thereto, ensuring optimum mutual positioning and facilitating the formation of a scar which is compatible with such arrangement.

Positively, the cosmetic product for the treatment of cicatricial tissues has an evident antibacterial function on the cicatricial tissues and on the tissues that are directly proximate thereto, since cyanoacrylate is characterized by known bactericidal functions.

Conveniently, the cosmetic product for the treatment of cicatricial tissues is capable of waterproofing completely the cicatricial tissues and the tissues that are directly proximate thereto, ensuring that the patient can get wet without the risk of residues and/or water (or other substances) infiltrating and reaching the vicinity of the scar.

Favorably, the cosmetic product for the treatment of cicatricial tissues is capable of maintaining optimum levels of humidity of the cicatricial tissues and of the tissues that are directly proximate thereto.

Just as favorably, the cosmetic product for the treatment of cicatricial tissues is capable of maintaining optimum temperature levels of the cicatricial tissues and of the tissues that are directly proximate thereto.

Therefore, the cosmetic product for the treatment of cicatricial tissues is capable of creating a microenvironment which is favorable for healing and in which there is minimal exchange with the outside at the cicatricial tissues and at the tissues that are directly proximate thereto.

Advantageously, as specifically noted earlier, the cosmetic product for the treatment of cicatricial tissues is capable of protecting against impacts the cicatricial tissues and the tissues that are directly proximate thereto, constituting a sort of mechanically rigid protective dome.

Positively, the cosmetic product for the treatment of cicatricial tissues is capable of dehydrating tissues during the heating caused by polymerization, eliminating thermolabile toxins and bacteria.

It has thus been found that the invention achieves the intended aim and objects.

The invention thus conceived is susceptible of numerous modifications and variations, all of which are within the scope of the appended claims.

All the details may further be replaced with other technically equivalent ones.

In the exemplary embodiments shown, individual characteristics, given in relation to specific examples, may actually be interchanged with other different characteristics that exist in other exemplary embodiments.

Moreover, it is noted that anything found to be already known during the patenting process is understood not to be claimed and to be the subject of a disclaimer.

The embodiment of the present invention shall be carried out in the most scrupulous compliance with the statutory and regulatory provisions related to the products of the invention or correlated thereto and following any required authorization of the corresponding competent authorities, with particular reference to regulations related to safety, environmental pollution and health.

In practice, the materials used, as well as the shapes and dimensions, may be any according to requirements without thereby abandoning the scope of the protection of the appended claims.

What is claimed is:

1-17. (canceled)

18. Use of at least one cyanoacrylate as sole active ingredient to prepare a cosmetic product for the treatment of cicatricial tissues, formed following healing of a tissue lesion, by application on the tissue lesion already healed.

19. The use according to claim 18, wherein said at least one cyanoacrylate is a cyanoacrylate derivative which has a hydrocarbon radical.

20. The use according to claim 19, wherein said hydrocarbon radical comprises 1 to 12 carbon atoms.

21. The use according to claim 20, wherein said hydrocarbon radical is a C1-C4-alkyl radical.

22. The use according to claim 20, wherein said product comprises butyl cyanoacrylate.

23. The use according to claim 20, wherein said product comprises methyl cyanoacrylate.

24. The use according to claim 20, wherein said product comprises propyl cyanoacrylate.

25. The use according to claim 20, wherein said product comprises isobutyl cyanoacrylate.

26. The use according to claim 20, wherein said product comprises ethyl cyanoacrylate.

27. The use according to claim 18, wherein said cyanoacrylate is an alkoxy-alkyl cyanoacrylate.

28. The use according to claim 27, wherein said alkoxy-alkyl cyanoacrylate is methoxy propyl cyanoacrylate.

29. The use according to claim 18, wherein said product further comprises dilution solvents.

30. The use according to claim 18, wherein it further comprises substances for adjusting viscosity.

31. The use according to claim 18, wherein it further comprises substances for adjusting thixotropy.

32. A cosmetic product for the treatment of cicatricial tissues, formed following healing of a tissue lesion, consisting of at least one cyanoacrylate and cosmetically acceptable excipients.

33. The product according to claim 32, further comprising one or more among dilution solvents, viscosity adjustment substances, and thixotropy adjustment substances.

34. A method for the cosmetic treatment of cicatricial tissues, formed following healing of a tissue lesion, comprising the step of applying to a tissue lesion already healed a cosmetic product according to claim 32.
